(19) United States
(12) Patent Application Publication
(10) Pub. No.: US 2023/0383088 A1
(43) Pub. Date: Nov. 30, 2023

(54) METHOD FOR DEPOLYMERIZING POLYMER MASSES WHILE DEGRADING ORGANIC HALOGEN COMPOUNDS

(71) Applicant: INEOS STYROLUTION GROUP GMBH, Frankfurt am Main (DE)

(72) Inventors: Norbert NIESSNER, Friedelsheim (DE); Bianca WILHELMUS, Hanau (DE); Hannes KERSCHBAUMER, Bad Soden am Taunus (DE); Hans-Werner SCHMIDT, Bayreuth (DE); Tristan KOLB, Bayreuth (DE); Andreas SCHEDL, Bayreuth (DE)

(21) Appl. No.: 18/249,479

(22) PCT Filed: Oct. 14, 2021
(86) PCT No.: PCT/EP2021/078426

§ 371 Date: Apr. 18, 2023

(30) Foreign Application Priority Data
Oct. 19, 2020 (EP) 20202633.2

(51) Int. Cl. C08J 11/12; C08J 11/16; C08L 25/06; C08K 5/02; C08K 3/22; C07C 4/22

(52) U.S. Cl. CPC C08J 11/12; C08J 11/16; C08L 25/06; C08K 5/02; C08K 3/22; C07C 4/22; C08J 2325/06; C08J 2333/12; C08K 2003/2217; C08K 2003/2206

(57) ABSTRACT

The invention relates to a process for depolymerization of polymer compositions with decomposition of halogen-containing organic compounds, wherein the polymer composition contains at least one styrene-containing polymer and at least one halogen-containing organic compound. The addition of one or more inorganic basic compounds in the depolymerization simultaneously promotes the decomposition of the halogen-containing organic compound and favors the depolymerization.

The present invention relates to a process for reducing the content of organic halogen compounds in the depolymerization (decomposition) of a polymer composition containing organic halogen compounds in the presence of basic inorganic compounds. A process for recovering styrene monomers from a polymer composition (A) by pyrolysis of a styrene-containing composition with decomposition of the halogen-containing compounds present therein have proven attractive for conservation of resources.

The transition from a linear to a circular economy is necessary both ecologically and economically from the viewpoints of climate change, environmental pollution, population growth and dependence on resources. As early as in the 1990s, intense efforts were directed towards the development of processes for the raw-material reuse of plastics wastes, but no industrial applications have arisen to date due to unresolved process-technological problems and for economic reasons. Due to generally increasing environmental awareness and the rising need for sustainable solutions, however, interest in chemical recycling is growing as well.

Not all thermoplastic polymers are equally well suited to chemical recycling. The thermal decomposition of polyolefins or polyesters forms mixtures of, inter alia, waxes, light oil and gases. Decomposition of polyethylene terephthalate (PET) results in organic acids, mainly benzoic acid and terephthalic acid, which are corrosive and may also cause blockage of the reactor (G. Grause et al., Feedstock recycling of waste polymeric material,Journal of Material Cycles and Waste Management, 13(4), 2011, 265-282).

In the case of polystyrene and other styrene-containing polymers/polymer compositions it is possible to depolymerize these polymers into their base constituents, especially styrene monomers, and for this reason polystyrene and other styrene monomer-containing polymers represent excellent starting materials for chemical recycling.

One technical problem is that a number of styrene-containing polymer compositions are provided with flame retardants, for example foams composed of expanded polystyrene (EPS) which are employed as insulating material in construction.

Impact modified polystyrene (HIPS), as used in electricals housings, or other styrene (co)polymers may also be admixed with halogen-containing additives.

The use of flame retardants is intended to reduce the risk of ignition and flame propagation in the event of a fire. 1,2,5,6,9,10-hexabromocyclododecane (HBCD) has historically been used as a flame retardant for EPS and HIPS. HBCD was included in the Stockholm Convention on Persistent Organic Pollutants in May 2013. There is a global ban on the manufacture and use of HBCD as a flame retardant. However, HBCD is still widespread in older buildings and electricals. When these products are sent for disposal it is necessary to make the HBCD present therein harmless or to remove it from the result of recycling (recyclate) in case the polymer is to be reused for new products.

HBCD-containing products have hitherto typically been sent for thermal recovery since there are no established processes to remove HBCD from the recyclate to such an extent that the statutory values are not exceeded.

Japanese publication JP-B 3752101 relates to a process for separating a thermoplastic resin composition into flame retardant and the thermoplastic resin. This process therefore covers a process for treating a flame retardant-containing thermoplastic resin composition, comprising the steps of: dispersing a thermoplastic styrenic polymer composition containing a bromine-based flame retardant in a solvent to dissolve at least a portion of the thermoplastic resin, successively removing at least a portion of the flame retardant of the thermoplastic resin from the solution in which the resin is dissolved and further removing at least a portion of the thermoplastic resin or of the flame retardant from the solution from which the flame retardant or the thermoplastic resin was removed.

U.S. Pat. No. 6,388,050 relates to a process for treating a flame retardant-containing styrenic resin composition, comprising the steps of: a dissolving or dispersing step (a) in which a styrenic resin composition containing a bromide flame retardant is contacted with a single solvent to dissolve or disperse at least a portion of the flame retardant, a separating step (b) in which a solution or dispersion of the flame retardant according to step (a) is separated, a drying step (c) in which the styrenic resin composition from which the flame retardant is separated according to step (b) is dried.

U.S. Pat. No. 7,435,772 describes the treatment of a resin composition containing a brominated flame retardant and an antimony-containing flame retardant with two solvents at a temperature between the glass transition temperature of the polymer and the boiling point of the respective solvent in two consecutive steps.

CN-A 105237799 relates to a process for separating polybrominated diphenyl ethers from polymer compositions using a solvent.

U.S. Pat. No. 8,138,232 describes a process for recycling a composition of at least two styrene-based polymers or copolymers in which the composition is mixed with a solvent and a precipitant is subsequently added.

JP-A 2016010906 relates to a process for removing HBCD from a foamed polystyrene composition comprising two solvents, one which dissolves HBCD but not polystyrene and a second which dissolves polystyrene.

The aforementioned publications all describe depleting the halogenated flame retardant through the use of a solvent. However, the brominated flame retardant is not made harmless or entirely removed.

JP-A 2000290424 relates to a process for reusing a thermoplastic resin composition containing a bromine-based flame retardant, wherein the resin composition is contacted with water or an alcohol to accelerate that debromination reaction of the flame retardant to remove the bromine. This process for reusing the thermoplastic resin composition which contains the bromide-based flame retardant additionally comprises contacting the resin composition with a metal hydroxide, a metal carbonate or octyl alcohol to produce the corresponding bromide salts or octyl bromide and subsequently eliminating the bromides produced or the octyl bromide produced from the resin.

U.S. Pat. No. 6,903,242 relates to a process for dehalogenating a halogen-containing flame retardant-resin composition which comprises a step in which the halogen-containing flame retardant-resin composition is contacted with a material mixture containing a dehalogenation material and a dehalogenation-promoting material at a temperature below the thermal decomposition temperature of the resin composition by kneading of the mixture and simultaneously application of shear force in a biaxial kneading extruder, a kneader or a roller mill.

DE-A 10 2016 125 506 relates to a process for reusing EPS foams comprising halogen-containing flame retardants, wherein the EPS foams are extruded, cooled and further comminuted into particles in the presence of other starting materials. The extrusion step is carried out in the presence of at least one halogen scavenger.

EP-A 2839863 relates to a process for deactivating brominated compounds using UV radiation without the use of a solvent.

What the abovementioned publications have in common is that the employed halogen scavengers usually only ensure deactivation of brominated flame retardants. The newly formed bromine compounds, for example CaBr₂, remain in the product and can thus influence the product properties or require costly and complex removal from the product.

Calcium compounds are known to react with halogen compounds from polymer compositions, for example CaCO₃ and CaO can react with the chlorine produced during pyrolysis of polyvinyl chloride (PVC) (K. Ragaert et al., Mechanical and chemical recycling of solid plastic waste, Waste Management, 69, 2017, 24-58). CaO and Ca(OH)₂ can likewise react with the bromide compounds formed during pyrolysis of a composition of polystyrene and brominated flame retardants (S. H. Jung et al., Fast pyrolysis of a waste fraction of high impact polystyrene (HIPS) containing brominated flame retardants in a fluidized bed reactor: The effects of various Ca-based additives (CaO, Ca(OH)₂ and oyster shells) on the removal of bromine, in: Fuel, 95, 2012, 514-520).

Alkali metal and alkaline earth oxides such as MgO, CaO, BaO and K₂O, in addition to zeolites SiO₂/Al₂O₃ and other solid acids and bases, are known to promote decomposition of polystyrene during pyrolysis (Z. Zhang et al., Chemical Recycling of Waste Polystyrene into Styrene over Solid Acids and Bases, Industrial & Engineering Chemistry Research, 34, 1995, 4514-4519).

JP 2545748 describes the use of metal oxides as catalyst for the decomposition of polystyrene. However, flame-retarded polystyrene is not described.

Zeolites and iron-laden zeolites are particularly effective bromine scavengers in the pyrolysis of compositions of polystyrene and brominated flame retardant (H. Wu et al., Fuel Oil Production from Two-Stage Pyrolysis-Catalytic Reforming of Brominated High Impact Polystyrene Using Zeolite and Iron Oxide Loaded Zeolite Catalysts, Open Journal of Ecology, 05(04), 2015, 136-146).

When polystyrene is subjected to sufficient thermal treatment it decomposes into styrene monomers. Incomplete decomposition also results in formation of, for example, styrene dimers, trimers and other oligomers. If the decomposition conditions are too harsh, byproducts such as for example benzene, toluene, ethylbenzene, cumene and alpha-methylstyrene may be formed. The amounts of these reaction products may vary. They depend in particular on the reaction conditions and the raw materials used (see C. Bouster et al., Study of the pyrolysis of polystyrenes: Kinetics of thermal decomposition, Journal of Analytical and Applied Pyrolysis, 1 (1980) 297-313 and C. Bouster et al., Evolution of the product yield with temperature and molecular weight in the pyrolysis of polystyrene, Journal of Analytical and Applied Pyrolysis 15 (1989) 249-259).

The styrene monomers obtained may optionally used for a new polymerization process. However, styrene oligomers can interfere with the polymerization process since even small amounts thereof affect important properties of the polymer. This also applies to other byproducts. Therefore, the styrene monomers must be separated from other components of the product mixture in order to ensure a high product quality.

EP-A 3635043 (INEOS Styrolution) describes a process for recovering styrene from styrene-containing plastic wastes by depolymerization. However, this process does not seek to deplete halogenated impurities.

There is presently a great need for a process, performable on a large scale, for producing styrene monomers from flame-retarded styrene-containing polymer compositions in a purity which meets the statutory provisions in terms of the content of halogen-containing flame retardants.

It is accordingly an object of the present invention to provide a process for removing halogen compounds or for rendering them harmless in the pyrolysis of a styrene-containing polymer/a polymer composition in the presence of a halogenated flame retardant.

It is a further object of the present invention to provide a process for reusing styrene-containing plastics comprising halogen-containing flame retardants in which the resulting product meets the statutory provisions in terms of the content of halogen-containing flame retardants and in which the physical properties of the resulting product are not adversely affected by the byproducts remaining in the product.

The present invention also relates to a process for reusing styrene-containing plastics comprising halogen-containing flame retardants, wherein the styrene-containing polymers are depolymerized in a reactor and the depolymerization step is carried out in the presence of at least one halogen scavenger.

The inventors of the present invention have found that such a process makes it possible to achieve the specified objects in technically practicable fashion.

It has surprisingly been found that basic inorganic oxides such as BaO, CaO and MgO favor both the depolymerization of styrene-containing polymer compositions and the decomposition of brominated flame retardants. Separating the gaseous reaction product from the reaction mixture makes it possible to achieve a reduction in the amount of brominated compounds in the product.

The present invention accordingly provides a process for recovering styrene monomers from a polymer composition (A) by pyrolysis with decomposition of the halogen-containing compounds present therein, comprising the steps of:
a) introducing:
   I) 25% to 99.95% by weight, based on the total weight of (A) and (B), of a polymer composition (A) comprising
      A1) 30% to 99.9999% by weight, based on the total weight of the polymer composition (A), of at least one styrene-containing polymer (A1) comprising
         Ia) 10% to 100% by weight, based on the total weight of the styrene-containing polymer (A1), of repeating units (Ia) deriving from styrene; and
         Ib) 0% to 90% by weight, based on the total weight of the styrene-containing polymer (A1), of rubber and/or of repeating units (Ib) which are distinct from the repeating units (Ia) and do not interrupt the pyrolysis process, preferably selected from repeating units (Ib) derived from acrylonitrile, acrylic esters, methyl methacrylate or alpha-methylstyrene or combinations thereof;
      A2) 0% to 69.9999% by weight, based on the total weight of the polymer composition (A), of further polymers (A2) which are distinct from the at least one styrene-containing polymer (A1) and do not interrupt the pyrolysis process, preferably selected from the group consisting of polyolefins, polycarbonates, polyesters, polyamides, polyalkyl (meth)acrylates, polyurethanes and combinations thereof, particularly preferably selected from the group consisting of polyethylene, polypropylene, polymethyl methacrylate and combinations thereof, particularly preferably polymethyl methacrylate;
      A3) 0.0001% to 50% by weight, based on the total weight of the polymer composition (A), of at least one halogen-containing compound (A3); and
      A4) 0% to 50% by weight, based on the total weight of the polymer composition (A), of further components (A4) which do not interrupt the pyrolysis process, preferably selected from the group consisting of fillers, stabilizers, other organic and/or inorganic additives, moisture and other organic and/or inorganic impurities; and
   II) 0.05% to 75% by weight, based on the total weight of (A) and (B), of at least one inorganic basic compound (B), into the reaction zone (R) of a pyrolysis reactor (P);
b) thermally cleaving the styrene-containing polymer (A1) present in the polymer composition (A) in the reaction zone (R) of the pyrolysis reactor (P) to obtain a product mixture (G) containing styrene monomers and further components;
c) withdrawing the product mixture (G) obtained in step b) from the reaction zone (R) of the pyrolysis reactor (P);
d) cooling the product mixture (G) withdrawn in step c) to obtain a condensed product mixture (G) containing styrene monomers and further components; and
e) separating a styrene monomer-containing fraction from the further components of the condensed product mixture (G) obtained in step d) to obtain a styrene monomer-containing liquid.

The thermal decomposition (depolymerization) of the polymer composition (A) may in principle be carried out in any reactor suitable as a pyrolysis reactor (P) in which the temperature required for decomposition can be achieved. For example, the pyrolysis reactor (P) may be selected from the group consisting of extruders, batch reactors, rotary tubes, microwave reactors, shell and tube reactors, vortex reactors and fluidized bed reactors.

The thermal decomposition may be performed in a rotary kiln for example. Rotary kilns are described for example in EP-A 1481957. The thermal decomposition may also be carried out in extruders; these are described for example in EP-A 1966291. Such reactors can be operated with or without a gas stream, such as carrier gas or gas as reaction medium.

The pyrolysis reactor (P) can accordingly be any known type of pyrolysis reactor, with the proviso that the construction of the pyrolysis reactor allows a precise adjustment of the temperature in the reaction zone (R) of the pyrolysis reactor (P).

The pyrolysis reactor (P) is preferably selected from the group consisting of twin-screw extruders, continuous stirred tank reactors, vortex reactors and fluidized bed reactors. If the pyrolysis reactor is a fluidized bed reactor it may be advantageous if the fluidized bed wholly or partially consists of the at least one inorganic basic compound (B).

Adjustment of the temperature in the reaction zone (R) of the pyrolysis reactor (P) may be carried out in any desired fashion. Temperature adjustment may be carried out in known fashion by microwave irradiation, using heat exchangers, gas burners, resistive heating conductors (resistance heating) or through introduction of superheated gas, in particular steam, in each case alone or in combination. In one embodiment adjustment of the temperature is carried out using resistive heating conductors which are in contact with the wall of the reaction zone (R) of the pyrolysis reactor (P).

Adjustment of the temperature may alternatively be carried out using steam which is provided through evaporation of water and brought to the desired temperature via a steam superheater.

The temperature in the reaction zone (R) of the pyrolysis reactor (P) is preferably adjusted by heating the reaction zone (R) through microwave-assisted heating.

The temperature in the reaction zone (R) of the pyrolysis reactor (P) is preferably adjusted to a temperature of 250° C. to 1000° C., particularly preferably of 300° C. to 700° C., very particularly preferably of 380° C. to 650° C., especially preferably of 400° C. to 600° C.

The introducing of the polymer composition (A) and the at least one inorganic basic compound (B) into the reaction zone (R) of the pyrolysis reactor (P) in step a) may be carried out in any desired manner. The introducing may be carried out by manual or mechanized addition of the components into the reaction zone (R) for example or by pneumatic feeding of the components into the reaction zone (R).

In a further embodiment the components are compounded with one another before being introduced into the reaction zone (R). This is particularly advantageous if the polymer composition (A) is to be subjected to a melt filtration before introduction into the reaction zone (R).

In a preferred embodiment the organic basic compound (B) is compounded with only a portion of the polymer composition (A) and introduced into the reaction zone (R) with a further amount of polymer composition (A) as a masterbatch.

The introducing of the components may be carried out continuously or discontinuously, i.e. by constant feeding or in one or more portions. The introducing of the polymer composition (A) and the at least one inorganic basic compound (B) into the reaction zone (R) of the pyrolysis reactor (P) is preferably carried out continuously, particularly preferably at a rate which compensates the consumption of the respective component during pyrolysis.

The polymer composition (A) is introduced into the reaction zone (R) of the pyrolysis reactor (P) in an amount of 25% to 99.95% by weight, preferably in an amount of 50% to 99.9% by weight, particularly preferably in an amount of 80% to 99.5% by weight, very particularly preferably in an amount of 90% to 99% by weight, especially preferably in an amount of 92% to 98% by weight, based on the total weight of (A) and (B).

Correspondingly the at least one inorganic basic compound (B) is introduced into the reaction zone of the pyrolysis reactor (P) in an amount of 0.05% to 75% by weight, preferably in an amount of 0.1% to 50% by weight, particularly preferably in an amount of 0.5% to 20% by weight, very particularly preferably in an amount of 1% to 10% by weight, especially preferably in an amount of 2% to 8% by weight, based on the total weight of (A) and (B).

In one embodiment the process may employ not only the polymer composition (A) and the at least one inorganic basic compound (B) but also further components which do not interrupt or interfere with the process, in particular processing aids. In a further embodiment the process employs no further components in addition to the polymer composition (A) and the at least one inorganic basic compound (B).

The polymer composition (A) employed in the process according to the invention often contains
A1) 30% to 99.9999% by weight, preferably 50% to 99.999% by weight, particularly preferably 70% to 99.99% by weight, very particularly preferably 90% to 99.9% by weight, based on the total weight the polymer composition (A), of at least one styrene-containing polymer (A1);
A2) 0% to 69.9999% by weight, preferably 0% to 49.999% by weight, particularly preferably 0% to 29.99% by weight, very particularly preferably 0% to 9.9% by weight, based on the total weight of the polymer composition (A), of further polymers (A2) which are distinct from the at least one styrene-containing polymer (A1) and do not interrupt the pyrolysis process;
A3) 0.0001% to 50% by weight, preferably 0.001% to 20% by weight, particularly preferably 0.01% to 10% by weight, very particularly preferably 0.1% to 5% by weight, based on the total weight of the polymer composition (A), of at least one halogen-containing compound (A3); and
A4) 0% to 50% by weight, preferably 0% to 30% by weight, particularly preferably 0% to 10% by weight, very particularly preferably 0% to 5% by weight, based on the total weight of the polymer composition (A), of further components (A4) which do not interrupt the pyrolysis process.

Accordingly the amount of at least one halogen-containing compound (A3) in the polymer composition (A) is at least 1 ppm, preferably at least 10 ppm, particularly preferably at least 100 ppm, very particularly preferably at least 1000 ppm.

In one embodiment the polymer composition (A) consists of the at least one styrene-containing polymer (A1), the at least one halogen-containing compound (A3), optionally further polymers (A2) and optionally further components (A4). In a further embodiment the polymer composition (A) consists only of the at least one styrene-containing polymer (A1) and the at least one halogen-containing compound (A3).

The at least one styrene-containing polymer (A1) is a polymer comprising
Ia) 10% to 100% by weight, preferably 30% to 100% by weight, particularly preferably 50% to 100% by weight, very particularly preferably 70% to 100% by weight, based on the total weight of the styrene-containing polymer (A1), of repeating units (Ia) deriving from styrene; and
b) 0% to 90% by weight, preferably 0% to 70% by weight, particularly preferably 0% to 50% by weight, very particularly preferably 0% to 30% by weight, based on the total weight of the styrene-containing polymer (A1), of rubber and/or of repeating units (Ib) which are distinct from the repeating units (Ia) and do not interrupt the pyrolysis process.

The rubber, if present in the styrene-containing polymer (A1), may be selected for example from the group consisting of diene rubber, acrylate rubber, ethylene-propylene-diene rubber (EPDM rubber), silicone rubber, natural rubber, composite rubbers thereof and combinations of the aforementioned rubbers. The rubber, if present, is preferably selected from butadiene rubber, isoprene rubber, butyl acrylate rubber, composite rubbers thereof, and combinations of these rubbers. The rubber, if present, is particularly preferably selected from butadiene rubber and n-butyl acrylate rubber. When rubber is present in the styrene-containing polymer (A1) the amount thereof is often at least 0.01% by weight based on the total weight of the styrene-containing polymer (A1).

The repeating units (Ib), if present in the styrene-containing polymer (A1), derive from comonomers such as for example acrylonitrile, vinyl chloride, alkyl acrylates, alkyl methacrylates, alpha-methyl styrene, maleic anhydride, phenylmaleimide, butadiene, ethylene, alpha-olefins or combinations thereof. The repeating units (Ib), if present in the styrene-containing polymer (A1), preferably derive from comonomers selected from the group consisting of acrylonitrile, acrylic esters, methyl methacrylate, alpha-methylstyrene and combinations thereof. If repeating units (Ib) are present in the styrene-containing polymer (A1) the amount thereof is at least 1% by weight, preferably at least 5% by weight, particularly preferably at least 10% by weight, very particularly preferably at least 20% by weight, based on the total weight of the styrene-containing polymer (A1). The styrene-containing polymer (A1) preferably contains no repeating units (Ib). If repeating units (Ib) are present in the styrene-containing polymer (A1) the amount thereof is often at least 0.01% by weight based on the total weight of the styrene-containing polymer (A1).

The styrene-containing polymer (A1) is preferably selected from the group consisting of styrene homopolymers (polystyrene), styrene copolymers such as styrene-acrylonitrile copolymers, styrene-alpha-methylstyrene copolymers, styrene-methyl methacrylate copolymers, styrene-alpha-methylstyrene-methyl methacrylate copolymers, styrene-alpha-methylstyrene-acrylonitrile copolymers, styrene-acrylonitrile-maleic anhydride copolymers, styrene-acrylonitrile-phenylmaleimide copolymers and graft copolymers thereof with rubber-like polymers such as acrylonitrile-butadiene-styrene graft copolymers (ABS), acrylonitrile-styrene-alkyl (meth)acrylate graft copolymers (ASA), styrene-alpha-methylstyrene-acrylonitrile-methyl methacrylate copolymers, styrene-alpha-methylstyrene-acrylonitrile-t-butyl methacrylate copolymers and styrene-acrylonitrile-t-butyl methacrylate copolymers.

The further polymers (A2), if present in the polymer composition (A), are distinct from the at least one styrene-containing polymer (A1) and do not interrupt the pyrolysis process. They are selected for example from the group consisting of thermosets, resins, polyolefins, polycarbonates, polyesters, polyamides, polyalkyl (meth)acrylates, polyurethanes, poly-alpha-methylstyrene, alpha-methylstyrene-methyl methacrylate copolymers, alpha-methylstyrene-acrylonitrile copolymers (AMSAN) and the graft copolymers thereof with rubber-like polymers such as alpha-methylstyrene-acrylonitrile-methyl methacrylate copolymers, alpha-methylstyrene-acrylonitrile-t-butyl methacrylate copolymers, acrylonitrile-butadiene-alpha-methylstyrene graft copolymers and combinations thereof. The further polymers (A2), if present in the polymer composition (A), are preferably selected from the group consisting of polyolefins, polycarbonates, polyesters, polyamides, polyalkyl (meth)acrylates, polyurethanes and combinations thereof, particularly preferably selected from the group consisting of polyethylene, polypropylene, polymethyl methacrylate and combinations thereof, very particularly preferably polymethyl methacrylate. If further polymers (A2) are present in the polymer composition (A) the amount thereof is often at least 0.01% by weight based on the total weight of the polymer composition (A).

Suitable polyolefins, if present, include any desired polyolefins such as for example polyethylene or polypropylene derivatives such as PE-LD (low-density polyethylene), PE-LLD (linear low-density polyethylene), PE-HD (high-density polyethylene), metallocene polyethylenes, ethylene copolymers such as poly(ethylene-co-vinyl acetate), ethylene-butene, ethylene-hexene, ethylene-octene copolymers and also cycloolefin copolymers, homo- or copolymers of propylene, metallocene-catalyzed polypropylenes and copolymers of propylene with other comonomers known to those skilled in the art and mixtures thereof. Preferred polyolefins include homopolymers of ethylene, homopolymers of propylene, copolymers of ethylene and propylene and mixtures thereof.

Suitable polyesters, if present, include any polyesters, for example polycondensation products of dicarboxylic acids containing 4 to 16 carbon atoms with diols containing 2 to 8 carbon atoms or polycondensation products of hydroxycarboxylic acids containing 2 to 6 carbon atoms. These include, inter alia, polyalkylene adipates, such as polyethylene adipate and polybutylene adipate, polyalkylene terephthalates, such as polyethylene terephthalate and polybutylene terephthalate, polylactic acid, polyhydroxybutyrates, polycaprolactones and polyvalerolactones. Preferred polyesters, if present, include polyethylene terephthalate (PET) and polybutylene terephthalate (PBT), especially polyethylene terephthalate.

Suitable thermosets or resins, if present, include any desired thermosets or resins, for example phenolic resins, urea resins, melamine resins, alkyd resins or epoxy resins and/or their respective curing products.

In one embodiment the styrene-containing polymer (A1) is polystyrene and any further polymer (A2) is polymethyl methacrylate.

The halogen-containing compound (A3), which is preferably a halogen-containing organic compound (A3), is a halogen-containing flame retardant for example.

Examples of the halogen-containing flame retardant are brominated flame retardants and chlorinated flame retardants. Preferred brominated flame retardants are selected from the group consisting of polybrominated diphenyl ethers (penta-, octa-, deca-bromodiphenyl ether), decabromodiphenylethane (DBDPE), tetrabromobisphenol A (TBBPA), polybrominated biphenyls (PBB), 2,4,6-tribromophenol (TBP), 1,2-bis(2,4,6-tribromophenoxy)ethane (BTBPE) and 1,2,5,6,9,10-hexabromocyclododecane (HBCD) and also brominated styrene-butadiene copolymers (FR-122P) and brominated polystyrene. Preferred chlorinated flame retardants are selected from the group consisting of chlorinated paraffins and Mirex (1,1a,2,2,3,3a,4,5,5a,5b,6-dodecachloroacta-hydro-1,3,4-metheno-1H-cyclobuta[cd]pentalene). The halogenated compound (A3) is particularly preferably a brominated flame retardant, preferably hexabromocyclododecane (HBCD) and/or polybrominated styrene-butadiene copolymer.

The other components (A4), if present in the polymer composition (A), may be any substances that do not interrupt the pyrolysis process and are distinct from the halogen-containing compound (A3) and the polymers (A1) and (A2). The further components (A4) are often customary plastic additives and auxiliaries.

The further components (A4) are preferably selected from the group consisting of fillers, stabilizers, other organic and/or inorganic additives, moisture and other organic and/or inorganic impurities. If further components (A4) are present in the polymer composition (A) the amount thereof is often at least 0.001% by weight based on the total weight of the polymer composition (A).

The other components (A4) may be selected for example from the group consisting of antioxidants, UV stabilizers, peroxide destroyers, antistats, lubricants, demolding agents, halogen-free flame retardants, fillers or reinforcers (glass fibers, carbon fibers, graphite, etc), colorants, nucleating agents, antiblocking agents, processing aids, plasticizers, non-halogenated flame retardants and combinations of two or more thereof.

Examples of oxidation retarders and heat stabilizers include halides of metals of group I of the periodic table, for example sodium, potassium and/or lithium halides, optionally in conjunction with copper(I) halides, for example chlorides, bromides, iodides, sterically hindered phenols, hydroquinones, various substituted representatives of these groups and mixtures thereof in concentrations of up to 2% by weight based on the total weight of the polymer composition (A).

Suitable UV stabilizers which are generally present in amounts of up to 2% by weight based on the total weight of the polymer composition (A) include various substituted resorcinols, salicylates, benzotriazoles and benzophenones.

It is also possible for organic dyes such as nigrosin, pigments such as titanium dioxide, phthalocyanines, ultramarine blue and carbon black to be present as colorants in the polymer composition (A) and also fibrous and pulverulent fillers and reinforcing agents. Examples of the latter are carbon fibers, glass fibers, amorphous silica, calcium silicate (wollastonite), aluminum silicate, magnesium carbonate, kaolin, chalk, powdered quartz, mica and feldspar.

Nucleating agents that may be present include for example talc, calcium fluoride, sodium phenylphosphinate, aluminum oxide, silicon dioxide and nylon 22.

Lubricants and demolding agents which may generally be employed in amounts of up to 1% by weight based on the total weight of the polymer composition (A) may include for example long-chain fatty acids such as stearic acid or behenic acid, their salts (e.g. Ca stearate or Zn stearate) or esters (e.g. stearyl stearate or pentaerythritol tetrastearate) and also amide derivatives (e.g. ethylenebisstearylamide).

Mineral-based antiblocking agents may moreover be present in amounts of up to 0.1% by weight based on the total weight of the polymer composition (A). Examples include amorphous or crystalline silica, calcium carbonate or aluminum silicate.

Also possibly present as a processing aid are, for example, mineral oil, preferably medicinal white oil, in amounts of up to 5% by weight, preferably up to 2% by weight, in particular 0.1% to 2% by weight, based on the total weight of the polymer composition (A).

Examples of plasticizers include dioctyl phthalate, dibenzyl phthalate, butyl benzyl phthalate, hydrocarbon oils, N-(n-butyl)benzenesulfonamide and o- and p-tolylethylsulfonamide.

Any of the non-halogenated flame retardants known for the respective thermoplastics may moreover be present, in particular those based on phosphorus compounds.

The polymer composition (A) may further contain moisture and/or further inorganic and/or organic impurities, for example foodstuffs residues, cement residues or adhesive residues.

The polymer composition (A) employed according to the invention may optionally be pretreated in suitable fashion, for example in order to remove adherent contaminants such as for instance foodstuffs residues or dirt, moisture and impurities such as metals or other substances and composite materials.

This is advantageously effected in a pretreatment which may comprise one or more of the following steps, wherein the sequence of the steps is not specified and steps may also be repeated multiple times: manual impurity sorting, washing, comminution, automatic sorting in suitable plants. It is optionally also possible to convert polymer compositions which do not correspond to the polymer composition (A) into a polymer composition (A) employed according to the invention by such a process.

Preference is therefore also given to an embodiment of the process according to the invention in which a polymer composition which does not correspond to the polymer composition (A) is subjected to a pretreatment comprising one or more of the following steps, wherein the sequence of the steps is not specified and steps may also be repeated multiple times: manual impurity sorting, washing, comminution, automatic sorting in suitable plants, to thus obtain the polymer composition (A).

The inorganic basic compound (B) is in particular a compound containing at least one element selected from the group of K, Na, Ca, Ba, Mg, Sr, Al, Ti and Si. Inorganic basic compounds (B) comprising these elements typically have an acceptable reactivity towards the at least one halogen-containing compound (A3) or its decomposition products.

The inorganic basic compound (B) may be selected from the group consisting of oxides, hydroxides, carbonates or a combination of oxides and hydroxides of the aforementioned elements. Examples of inorganic basic compounds (B) are K 2 O, Na 2 O, NaOH, CaO, BaO, SrO, Ca(OH)₂, Ba(OH)₂, CaCO₃, MgO, Al₂O₃, SiO₂, TiO₂, Mg(OH) 2 and SrO. The inorganic basic compound is preferably selected from the group consisting of alkali metal oxides, alkaline earth metal oxides, alkali metal hydroxides, alkaline earth metal hydroxides and combinations thereof. The inorganic basic compound (B) is particularly preferably selected from the group consisting of MgO, CaO, SrO, BaO, K₂O and combinations thereof. The inorganic basic compound (B) is very particularly preferably selected from the group consisting of CaO, BaO and combinations thereof, in particular BaO.

Without wishing to be bound by any particular theory it is thought that the alkali or alkaline earth metal compound reacts with the halogen atom present in the halogen-containing flame retardant in order thus to form a stable halogen compound. Separation of such stable halogen compound may be carried out by customary physical separation processes. If organic halogen compounds are formed these are likewise separable from the reaction mixture, for example by distillation.

In process step a) the inorganic basic compound (B) may also be introduced into the reaction zone (R) of the pyrolysis reactor (P) separately from the remaining components of the inventive polymer composition (A), for example as a solid. In a preferred embodiment the inorganic basic compound (B) is introduced into the reaction zone (R) of the pyrolysis reactor (P) and in the course of the process wholly or partially continuously or discontinuously withdrawn from the reaction zone (R) and wholly or partially replaced by unused inorganic compounds (B).

In a particular embodiment the inorganic compound (B) may wholly or partially form the bed material in a fluidized bed reactor and be passed through and regenerated in a separate circuit. In a further particular embodiment, the inorganic, basic compound (B) is introduced into the reaction zone (R) in conjunction with other inorganic compounds, for example as a surface coating on a bed material. In a particular embodiment the bed material, which wholly or partially consists of the inorganic, basic compound (B), is heated under oxidizing conditions and thus regenerated after the reaction.

In one embodiment the weight ratio of the at least one inorganic basic compound (B) to the at least one halogen-containing compound (A3) is chosen such that it is in the range from 1:1 to 10:1, preferably in the range from 1.5:1 to 5:1, particularly preferably in the range 2:1 to 3:1.

It is preferable when no antimony oxide (Sb₂O₃) is present in the polymer composition (A) or in the inorganic basic compound (B), particularly preferably in the entire process according to the invention. It is very particularly preferable when no antimony compounds whatsoever are present in the process according to the invention. If such antimony compounds are present it may be advantageous to remove them from the polymer before step a). Without wishing to be bound by any particular theory, it is thought that the presence of antimony compounds, particularly antimony oxide, may have an adverse effect on the formation of styrene monomers during the pyrolysis of styrenic polymers.

After the introducing of the polymer composition (A) and the inorganic basic compound (B) into the reaction zone (R) of the pyrolysis reactor (P) in step a), step b) comprises thermally cleaving the styrene-containing polyme (A1) present in the polymer composition (A) in the reaction zone (R) of the pyrolysis reactor (P) to obtain a product mixture (G) containing styrene monomers and further components.

The thermal cleaving is preferably carried out at a temperature of 250° C. to 1000° C., preferably of 300° C. to 700° C., particularly preferably of 380° C. to 650° C., very particularly preferably of 400° C. to 600° C. The average residence time (Z) of the polymer composition (A) in the reaction zone (R) of the pyrolysis reactor is preferably 0.01 seconds to 21 600 s, particularly preferably 0.01 to 3600 s, very particularly preferably 0.01 to 500 seconds, especially preferably 0.1 to 5 s.

In a preferred embodiment the thermal cleaving is preferably carried out at a pressure of less than 1200 mbar, preferably at a pressure of less than 1013 mbar, particularly preferably at a pressure of less than 500 mbar, very particularly preferably at a pressure of less than 300 mbar.

During the residence time (Z) in the reaction zone (R) of the pyrolysis reactor (P) the styrene-containing polymer (A1) and optionally further polymers (A2) of the polymer composition (A) are at least partially depolymerized and the at least one halogen-containing compound (A3) and optionally further components (A4) which are sensitive to the selected conditions are at least partially decomposed, wherein the at least one halogen-containing compound (A3) and optionally its decomposition products react with the at least one basic inorganic compound (B) to form a product mixture (G) containing styrene monomers and other components.

The product mixture (G) containing styrene monomers and other components is withdrawn from the reaction zone (R) of the pyrolysis reactor (P) in step c). Withdrawal is preferably carried out continuously. It is particularly preferable when the product mixture (G) is continuously withdrawn from the upper region of the reaction zone (R) of the pyrolysis reactor (P) in the gaseous state. The withdrawal of the product mixture (G) may be carried out under reduced pressure. In a further preferred embodiment the withdrawal of the product mixture (G) may for example be effected automatically through transport of the product mixture (G) out of the reaction zone (R) due to the elevated pressure in the reaction zone (R) brought about by the reaction.

The pyrolysis reactor may contain a quench or be connected to a quench. In the present context a quench is to be understood as meaning a region of the pyrolysis reactor in which the depolymerization reaction is rapidly terminated, preferably by cooling the hot gas comprising the product mixture (G). The quench inter alia stabilizes the reaction products and prevents or reduces undesired repolymerization. Typical quenches cool the product mixture (G) from the temperature established in the reaction zone (R) of the pyrolysis reactor (P) to a temperature of less than 250° C. within a very short time, preferably within less than 10 seconds, particularly preferably within less than 5 seconds, particularly preferably within less than 1 second. Quenches are described in EP 1966291 for example.

The product mixture (G) is cooled in step d) after withdrawal from the reaction zone (R) of the pyrolysis reactor (P), as a result of which condensation of the styrene monomers and further components occurs and a condensed product mixture (G) containing styrene monomers and other components is obtained. The product mixture (G) is cooled to a temperature below the condensation point of styrene monomers. The product mixture (G) is preferably cooled to a temperature below 70° C., particularly preferably below 50° C., very particularly preferably below 40° C. In a preferred embodiment the product mixture (G) is cooled to a temperature of from −200° C. to 70° C. In a particularly preferred embodiment the product mixture (G) is cooled to a temperature of from −200° C. to 40° C. In a preferred embodiment the product mixture (G) is cooled to a temperature of −5° C. to 30° C.

In a further embodiment the product mixture (G) is cooled and condensed in a condensation apparatus having at least two temperature zones. In this case the product mixture (G) is cooled to a temperature of −30° C. to 50° C., preferably of −15° C. to 30° C., particularly preferably of −5° C. to 15° C., in a first temperature zone and cooled to a temperature of −200° C. to −50° C., preferably of −200° C. to −100° C., particularly preferably of −200° C. to −150° C. in a further temperature zone. In this embodiment the condensation is preferably carried out under reduced pressure.

The cooling can be effected in any known manner. For example, cooling on a solid surface which is cooled by water or air is possible. Likewise possible is cooling by means of a water mist which is brought directly into contact with the product mixture (G).

The cooling preferably takes place in a water mist. Here, the condensable constituents of the product mixture (G) are condensed according to their vapor pressures and collected together with the water. A condensed product mixture (G) containing styrene monomers and further constituents is obtained in the condensation. In the case of cooling in a water mist, the condensed product mixture (G) is obtained as a two-phase system with the cooling water. In this case, the condensed product mixture (G) is separated from the aqueous phase as a floating organic phase. The aqueous phase can be cooled again and used as water mist for cooling the product mixture (G).

The condensed product mixture (G′) typically contains more than 10% by weight, based on the total weight of the condensed product mixture (G′), of styrene monomers and less than 90% by weight, based on the total weight of the condensed product mixture (G), of the further constituents. The condensed product mixture (G′) preferably contains 10% to 99% by weight, based on the total weight of the condensed product mixture (G′), of styrene monomers and 1% to 90% by weight, based on the total weight of the condensed product mixture (G′), of the further constituents, particularly preferably 50% to 99% by weight, based on the total weight of the condensed product mixture (G′), of styrene monomers and 1% to 50% by weight, based on the total weight of the condensed product mixture (G′), of the further constituents, very particularly preferably 70% to 98% by weight, based on the total weight of the condensed product mixture (G′), of styrene monomers and 2% to 30% by weight, based on the total weight of the condensed product mixture (G′), of the further constituents.

The condensed product mixture (G′) moreover typically contains more than 40% by weight, preferably more than 50% by weight, particularly preferably more than 55% by weight, very particularly preferably more than 60% by weight, of styrene based on the total weight of the repeating units (Ia) derived from styrene in the at least one styrene-containing polymer (A1).

In the further course of the process step e) comprises separating the condensed product mixture (G′) into a styrene monomer-containing fraction and further fractions for example. This separation may be performed in any known manner which is suitable for separating mixtures of liquid products and possibly solids into their constituents.

Depending on the further constituents besides styrene monomers, suitable methods for separating the condensed product mixture (G′) into a styrene monomer-containing fraction and further fractions may comprise for example sedimentation, centrifugation, filtration, decantation, distillation, chromatography, crystallization and sublimation.

If solids are present in the condensed product mixture (G), it is advantageous to remove the solids prior to separation of the liquid constituents. In this case it is preferable to remove the solids from the liquid constituents by sedimentation, centrifugation, filtration, distillation, sublimation and/or decantation, particularly preferably by filtration, very particularly preferably by filtration using a solid-liquid filter.

The further separation of the liquid constituents into a styrene monomer-containing fraction and further fractions preferably comprises at least one step of distillation, such as for example fractional distillation, at least one step of chromatography, such as for example column chromatography, HPLC or flash chromatography, and/or at least one step of crystallization, such as for example fractional crystallization. The separation of the liquid constituents particularly preferably comprises at least one step of distillation, very particularly preferably at least one step of fractional distillation. In a particularly preferred embodiment the separation of the liquid constituents into a styrene monomer-containing fraction and further fractions comprises at least one step of fractional distillation in one or more rectifying columns. The styrene-monomer-containing fraction is collected as a styrene-monomer-containing liquid.

In a preferred embodiment the proportion of halogenated compounds in the styrene monomer-containing liquid is less than 1000 ppm, preferably less than 500 ppm, particularly preferably less than 100 ppm, based on the styrene monomer-containing liquid.

It is often advantageous to recycle at least a portion of the further fractions into the reaction zone (R) of the pyrolysis reactor (P). This is especially advantageous when a portion of the further functions contains styrene oligomers such as for example styrene dimers and styrene trimers. This makes it possible to achieve an altogether better yield of styrene monomers since this also enables depolymerization of the remaining oligomers.

It is therefore preferable for at least a portion of the further fractions of the condensed product mixture (G′) which have been separated from the styrene monomer-containing fraction to be recycled into the reaction zone (R) of the pyrolysis reactor (P). Particular preference is given to continuous recycling of the further fractions, wherein recycling of the fractions that are higher boiling than the styrene monomer-containing fraction is preferred since these contain the highest proportion of styrene oligomers

In a preferred embodiment the further components which are recycled into the reaction zone (R) of the pyrolysis reactor (P) essentially consist of styrene oligomers, preferably of styrene dimers and styrene trimers. In this context, “essentially” means that the further components which are recycled into the reaction zone (R) of the pyrolysis reactor (P) do not contain any further components which disrupt the depolymerization process in the reaction zone (R) of the pyrolysis reactor (P) besides styrene oligomers.

The invention further provides an apparatus for performing a process for producing styrene monomers from a styrene-containing polymer composition as described above, in which the reaction zone (R) and the pyrolysis reactor (P) in the apparatus are configured such that gentle depolymerization of styrene of the polymer composition (A) may be carried out.

The apparatus preferably comprises a pyrolysis reactor (P) having a reaction zone (R), at least one heating element for setting the desired temperatures, at least one conduit for introducing the polymer mass (A) into the reaction zone (R) and for setting the residence time (Z), and a quencher for cooling down the product mixture (G).

The present invention further provides the condensed product mixture (G′) containing styrene monomers and further components obtainable from step d) of the process according to the invention.

The invention likewise provides the styrene monomer-containing liquid obtainable by the process according to the invention. This liquid preferably contains less than 1000 ppm, preferably less than 500 ppm, particularly preferably less than 100 ppm, based on the total weight of the styrene monomer-containing liquid, of halogenated compounds.

The present invention further provides for the use of an inorganic basic compound (B) as described above for decomposition of halogen-containing organic compounds of monomers in the pyrolytic recovery of monomers from polymer compositions in the absence of antimony oxide (SbO₃), preferably from polymer compositions comprising styrene-containing polymers.

The invention is illustrated by the following examples and claims.

The experiments relating to the invention were performed in a batch process on the laboratory scale. To this end a modified Versoclave Typ 3E/2.0 litre laboratory stirred autoclave from Büchi AG (Uster, Schweiz) which is heatable to 500° C. via an electric heating jacket was employed. Temperature control was effected via temperature sensors in the jacket, in the reactor bottom and an immersion sensor in the melt introduced via the cover plate. The initially charged material was commixed during the depolymerization process via a stirring means fitted with an anchor stirrer. Via a discharging port in the cover plate of the reactor the volatile product mixture was condensed and collected in a glass condensation apparatus having two temperature zones (0° C. and −196° C.). The entire experimental apparatus consisting of a batch reactor and a condensation apparatus was evacuated via a downstream membrane pump.

Comparative experiment 2 and experiments 1 to 3 employed 98 parts of PS, two parts of HBCD and, in addition to experiments 1 to 3, five parts of an inorganic base. Employable inorganic bases include MgO (experiment 1), CaO (experiment 2) and BaO (experiment 3). Typical batch amounts are 245 g of PS, 5 g of HBCD and 12.5 g of the inorganic base. After charging with the reaction mixture the autoclave was sealed, evacuated and over several hours (typically 5 to 6 hours) heated to a target temperature of 380° C. (measured at the reactor bottom) with stirring and over two or more steps. Depolymerization of the reaction mixture started during the heating.

The volatile reaction products were condensed in fractions as described above. The resulting fractions were characterized by gas chromatography and elemental analysis to determine the styrene and halogen content. The gas chromatography analyses were performed using an Agilent 7890a gas chromatograph with a DB-1 column of 25 m in length (Agilent). Detection was by means of an FID detector and THF was used as solvent.

For comparative experiment 1, PS was depolymerized in a batch process. The batch amount was 500 g of PS. After charging with the reaction mixture the autoclave was sealed, evacuated and over several hours (5 hours) heated to 380° C. (measured at the reactor bottom) with stirring and over two or more steps. Towards the end of the experiment the temperature was increased to 450° C. (measured at the reactor bottom) for 10 minutes. Depolymerization of the reaction mixture started during the heating. The volatile products were condensed in fractions as described above. The resulting fractions were combined and characterized by gas chromatography.

The results are shown in Table 1. The reported condensate yield is the mass of the volatile compounds of all collected fractions relative to the sum of the employed mass of the reaction mixture. The styrene yield relates to the mass of the styrene in the reaction mixture. The bromine content is the percentage by mass of bromine in the overall condensate. The capture efficiency is calculated from the ratio of detected bromine content in the overall condensate to the bromine content in the employed reaction mixture. The mass content of bromine in HBCD is 74.71% by weight.

TABLE 1
Comparison of performed experiments

| | Compar- | Compar- | | | |
|---|---|---|---|---|---|
| | ative | ative | | | |
| | exper- | exper- | Exper- | Exper- | Exper- |
| | iment 1 | iment 2 | iment 1 | iment 2 | iment 3 |
| Proportion of PS | 100 | 98 | 98 | 98 | 98 |
| [parts by wt.] | | | | | |
| Proportion | 0 | 2 | 2 | 2 | 2 |
| of HBCD | | | | | |
| [parts by wt.] | | | | | |
| Proportion of base | 0 | 0 | 5 | 5 | 5 |
| [parts by wt.] | | | | | |
| Employed base | — | — | MgO | CaO | BaO |
| Condensate yield | 97.68 | 89.13 | 91.84 | 95.44 | 92.88 |
| [% by wt.] | | | | | |
| Styrene yield | 58.12 | 43.33 | 44.70 | 60.50 | 61.02 |
| [% by wt.] | | | | | |
| Bromine content in | — | 2.060 | 1.945 | 0.153 | 0.158 |
| condensate [g] | | | | | |
| Bromine content in | — | 3.735 | 3.735 | 3.735 | 3.735 |
| reactant [g] | | | | | |
| Capture efficiency | — | 44.8 | 47.9 | 95.9 | 95.8 |
| [% by wt.] | | | | | |

t is clearly apparent from the measured results that the addition of inorganic bases, in particular of the components CaO and/or BaO, result in a marked reduction in the bromine content in the condensate. The styrene yield is also good.

The described process may also be performed two or more times in succession and may lead to further depletion in halogen-containing compounds.

HBCD-containing polymer compositions result in a lower yield of styrene oil as is apparent from a comparison of the yields of comparative experiment 1 and comparative experiment 2. The addition of inorganic bases compensates for the yield losses in the depolymerization of flame retardant-containing polymer compositions or even overcompensates the yield losses, as is clearly apparent from experiments 1 to 3, in particular from experiments 2 and 3.

Analogous experiments are also performable with other styrene-containing polymers (as polystyrene).

1-21. (canceled)

22. A process for recovering styrene monomers from a polymer composition (A) by pyrolysis with decomposition of at least one halogen-containing compound present therein, comprising the steps of:
a) introducing:
I) 25% to 99.95% by weight, based on the total weight of the polymer composition (A) and at least one inorganic basic compound (B), of the polymer composition (A) comprising:
A1) 30% to 99.9999% by weight, based on the total weight of the polymer composition (A), of at least one styrene-containing polymer (A1) comprising:
Ia) 10% to 100% by weight, based on the total weight of the at least one styrene-containing polymer (A1), of repeating units (Ia) deriving from styrene; and
Ib) 0% to 90% by weight, based on the total weight of the at least one styrene-containing polymer (A1), of rubber and/or of repeating units (Ib) which are distinct from the repeating units (Ia) and do not interrupt the pyrolysis process;
A2) 0% to 69.9999% by weight, based on the total weight of the polymer composition (A), of further polymers (A2) which are distinct from the at least one styrene-containing polymer (A1) and do not interrupt the pyrolysis process;
A3) 0.0001% to 50% by weight, based on the total weight of the polymer composition (A), of at least one halogen-containing compound (A3); and
A4) 0% to 50% by weight, based on the total weight of the polymer composition (A), of further components (A4) which do not interrupt the pyrolysis process; and
II) 0.05% to 75% by weight, based on the total weight of the polymer composition (A) and the at least one inorganic basic compound (B), of at least one inorganic basic compound (B),
into a reaction zone (R) of a pyrolysis reactor (P);
b) thermally cleaving the at least one styrene-containing polymer (A1) present in the polymer composition (A) in the reaction zone (R) of the pyrolysis reactor (P) to obtain a product mixture (G) containing styrene monomers and further components;
c) withdrawing the product mixture (G) obtained in step b) from the reaction zone (R) of the pyrolysis reactor (P);
d) cooling the product mixture (G) withdrawn in step c) to obtain a condensed product mixture (G′) containing styrene monomers and further components; and
e) separating a styrene monomer-containing fraction from the further components of the condensed product mixture (G′) obtained in step d) to obtain a styrene monomer-containing liquid;
wherein no antimony oxide (Sb₂O₃) is present in the polymer composition (A) or in the at least one inorganic basic compound (B).

23. The process of claim 22, wherein process step b) is carried out at a temperature of 250° C. to 1000° C.

24. The process of claim 22, wherein an average residence time (Z) of the polymer composition (A) in the reaction zone (R) of the pyrolysis reactor (P) in step b) is from 0.01 seconds to 21,600 seconds.

25. The process of claim 22, wherein process step b) is carried out at a pressure of less than 1200 mbar.

26. The process of claim 22, wherein the separating of the styrene monomer-containing fraction in step e) is carried out by fractional distillation.

27. The process of claim 22, wherein the at least one halogen-containing compound (A3) is at least one organic halogen-containing flame retardant.

28. The process of claim 22, wherein the polymer composition (A) contains at least 10 ppm of the at least one halogen-containing compound (A3).

29. The process of claim 22, wherein the at least one inorganic basic compound (B) is selected from the group consisting of basic compounds of main group and transition group metals.

30. The process of claim 22, wherein the at least one inorganic basic compound (B) is selected from the group consisting of alkali metal oxides, alkaline earth metal oxides, alkali metal hydroxides, alkaline earth metal hydroxides, and combinations thereof.

31. The process of claim 22, wherein the at least one styrene-containing polymer (A1) is polystyrene and any further polymers (A2) present are polymethyl methacrylate.

32. The process of claim 22, wherein the at least one inorganic basic compound (B) is introduced into the reaction zone (R) of the pyrolysis reactor (P) independently of the polymer composition (A) and in the course of the process is wholly or partially, continuously or discontinuously withdrawn from the reaction zone (R) and wholly or partially replaced by at least one unused inorganic basic compound (B).

33. The process of claim 22, wherein the pyrolysis reactor (P) is a fluidized bed reactor and a fluidized bed of the fluidized bed reactor wholly or partially consists of the at least one inorganic basic compound (B).

34. The process of claim 22, wherein the pyrolysis reactor (P) is a twin-screw extruder, a continuous stirred tank reactor, or a vortex reactor.

35. The process of claim 22, wherein the reaction zone (R) of the pyrolysis reactor (P) is heated through microwave-assisted heating.

36. The process of claim 22, wherein no antimony compounds are present in the process.

37. The process of claim 22, wherein the condensed product mixture (G′) contains more than 40% by weight of styrene, based on the total weight of the repeating units (Ia) derived from styrene in the at least one styrene-containing polymer (A1).

38. A condensed product mixture (G′) containing styrene monomers and further components obtained from step d) of the process of claim 22.

39. A styrene monomer-containing liquid obtained by the process of claim 22.

40. The styrene monomer-containing liquid of claim 39, wherein the proportion of halogenated compounds is less than 1000 ppm, based on the total weight of the styrene monomer-containing liquid.

41. An apparatus for performing the process of claim 22, comprising a pyrolysis reactor (P) having a reaction zone (R), wherein the apparatus is configured such that gentle depolymerization of styrene of the polymer composition (A) is carried out.
